(19) United States
(12) Patent Application Publication
(10) Pub. No.: US 2021/0350884 A1
(43) Pub. Date: Nov. 11, 2021

(54) METHOD AND SYSTEM FOR CREATION OF A MEDICAL NOTE

(71) Applicant: NotePro, Inc., State College, PA (US)

(72) Inventor: Christine E. Weikert, State College, PA (US)

(73) Assignee: NotePro, Inc., State College, PA (US)

(21) Appl. No.: 17/308,189

(22) Filed: May 5, 2021

Related U.S. Application Data
(60) Provisional application No. 63/021,350, filed on May 7, 2020.

(51) Int. Cl. G16H 10/60; G06Q 10/10

(52) U.S. Cl. CPC G16H 10/60; G06Q 10/10

(57) ABSTRACT

A system of specialty-specific and condition-specific medical content that a clinician can use to create a medical note without typing or dictating, and that can be integrated into any electronic health record. By linking data throughout the medical note, the system creates the most specific clinical documentation, with the most precise diagnoses and treatments, while automatically tabulating the highest level of diagnostic and billing codes. The system can significantly simplify the creation of complex medical documentation with precise, automated coding for diagnoses and billing for physicians and medical institutions.

### BACKGROUND OF THE INVENTION

In electronic health records (EHR), most of the relevant information for diagnosis and billing are either typed or dictated by the clinician. After a medical appointment during which the clinician writes a medical note, a clinician or biller needs to manually choose the correct diagnosis codes from the 68,000 ICD10 codes, and the correct billing code from the 10,000 CPT codes. Because the coding process is complex, requiring not only the correct diagnosis and billing codes, but also the number of medical conditions, the degree of difficulty of decision-making during the clinical visit, the time spent, and other factors, most clinicians and billers spend an inordinate amount of time documenting and billing and consistently underbill to avoid an audit.

The transition to EHRs was supposed to move hospitals and medical institutions into the twenty-first century, but instead poor EHR architecture has caused inefficiencies that have placed even more burdens on the clinician. Creating accurate information for medical records and using it for diagnostics and billing is labor intensive and conducive to human error. A method that automatically determines the most accurate diagnostic and billing codes while the clinician creates the medical note eliminates the manual labor of finding the right diagnostic codes and matching the appropriate billing code. The method, a dynamic system for creating a specialty and condition-specific medical note that automatically tabulates the highest level of diagnostic and billing codes, eliminates human error and creates more efficiency in diagnostics and billing.

Various publications show that the medical content in existing EHRs is limited, generic, mostly template-based, and grossly inadequate. This leads to errors in diagnoses, increased patient morbidity and mortality, coding and billing errors, decreased revenue, and increased audit liability.

The documentation of the four sections of a medical note, which include the Chief Complaint (with History of Present Illness), the Physical Exam, the Diagnosis, and the Plan (including CPT codes), must be consistent and precise, otherwise the medical note provides poor documentation and the insurance companies will deny the claim.

### SUMMARY OF THE INVENTION

In some embodiments, a medical note system includes one or more processors; and one or more memories operatively coupled to at least one of the one or more processors and having instructions stored thereon that, when executed by at least one of the one or more processors, cause at least one of the one or more processors to: receive instructions for an initial prompt; display the initial prompt for a user to interact with the initial prompt; recognize selection of an initial data point, wherein the initial data point includes information relevant for determining diagnosis and billing; determine a diagnostic code matching the recognized initial data point; determine a second prompt based on selection of initial data point, wherein the second prompt relates to more specific information related to the initial data point coming from the initial prompt; display the second prompt; recognize selection of a second data point, wherein the second data point includes information relevant for determining diagnosis and billing; determine a second diagnostic code matching the recognized second data point, wherein the second diagnostic code relates to the more specific second data point; and create a medical note, wherein the medical note includes the second diagnostic code and sections, wherein the sections are structured data sets from the selection of data points; wherein the sections are interlinked to allow for a single selection to populate the structured data for multiple sections, wherein the interlinking is configured to provide specific diagnostic codes and specific billing codes. In some embodiments, the medical note includes the initial data point and the second data point. In some embodiments, the system includes auto-populating a third prompt based on the selection of a previous data point. In some embodiments, a determination of the second prompt is determined based on a designated intake mapping structure. In some embodiments, the system includes inputting the medical note into an electronic health records database. In some embodiments, the diagnosis codes are ICD 10 diagnostic codes. In some embodiments, the medical note is condition-specific.

In some embodiments, a medical note method includes: receiving instructions for an initial prompt; displaying the initial prompt for a user to interact with the initial prompt; recognizing selection of an initial data point, wherein the initial data point includes information relevant for determining diagnosis and billing; automatically determining a diagnosis code matching the recognized initial data point; automatically determining a second prompt based on selection of initial data point, wherein the second prompt relates to more specific information related to the initial data point coming from the initial prompt; displaying the second prompt; recognizing selection of a second data point, wherein the second data point includes information relevant for determining diagnosis and billing; automatically determining a second diagnosis code matching the recognized second data point, wherein the second diagnosis code relates to the more specific second data point; and automatically creating a medical note, wherein the medical note includes the second diagnosis code and sections, wherein the sections are structured data sets from the selection of data points; wherein the sections are interlinked to allow for a single selection to populate the structured data for multiple sections, wherein the interlinking is configured to provide specific diagnostic codes and specific billing codes. In some embodiments, the medical note includes the medical information. In some embodiments, the method includes auto-populating a third prompt based on the selection of a previous data point. In some embodiments, a determination of the second prompt is determined based on a designated intake mapping structure. In some embodiments, the method includes inputting the medical note into an electronic health records database. In some embodiments, the diagnosis codes are ICD 10 diagnostic codes. In some embodiments, the medical note is condition-specific.

In some embodiments, non-transitory computer-readable storage medium storing program instructions computer-executable to perform, includes receiving instructions for an initial prompt; displaying the initial prompt for a user to interact with the initial prompt; recognizing selection of an initial data point, wherein the initial data point includes information relevant for determining diagnosis and billing; determining a diagnosis code matching the recognized initial data point; determining a second prompt based on selection of initial data point, wherein the second prompt relates to more specific information related to the initial data point coming from the initial prompt; displaying the second prompt; recognizing selection of a second data point, wherein the second data point includes information relevant for determining diagnosis and billing; determining a second diagnosis code matching the recognized second data point, wherein the second diagnosis code relates to the more specific second data point; and creating a medical note, wherein the medical note includes the second diagnosis code and sections, wherein the sections are structured data sets from the selection of data points; wherein the sections are interlinked to allow for a single selection to populate the structured data for multiple sections, wherein the interlinking is configured to provide specific diagnostic codes and specific billing codes. In some embodiments, the medical note further comprises the medical information. In some embodiments, the non-transitory computer-readable storage medium storing program instructions computer-executable to perform includes auto-populating a third prompt based on the selection of a previous data point. In some embodiments, a determination of the second prompt is determined based on a designated intake mapping structure. In some embodiments, the non-transitory computer-readable storage medium storing program instructions computer-executable to perform includes inputting the medical note into an electronic health records database. In some embodiments, the diagnosis codes are ICD 10 diagnostic codes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 depicts an embodiment of a system of medical note creation.

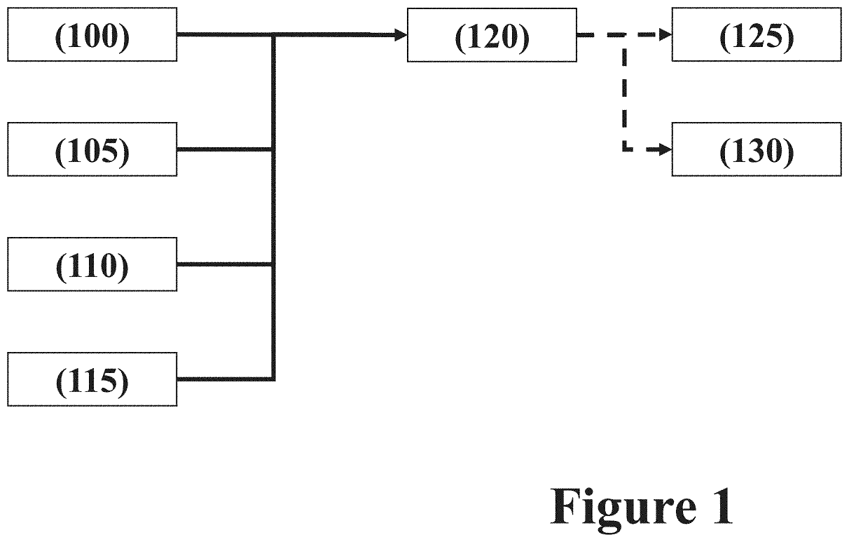

FIG. 2 depicts an exemplary embodiment of an examination document.

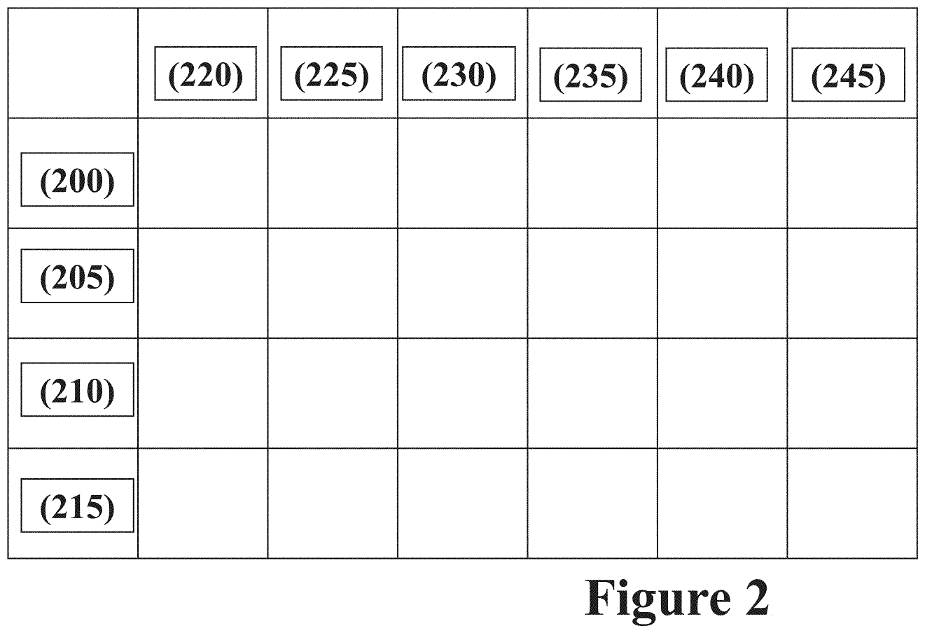

FIG. 3 illustrates an embodiment of a method for medical note creation.

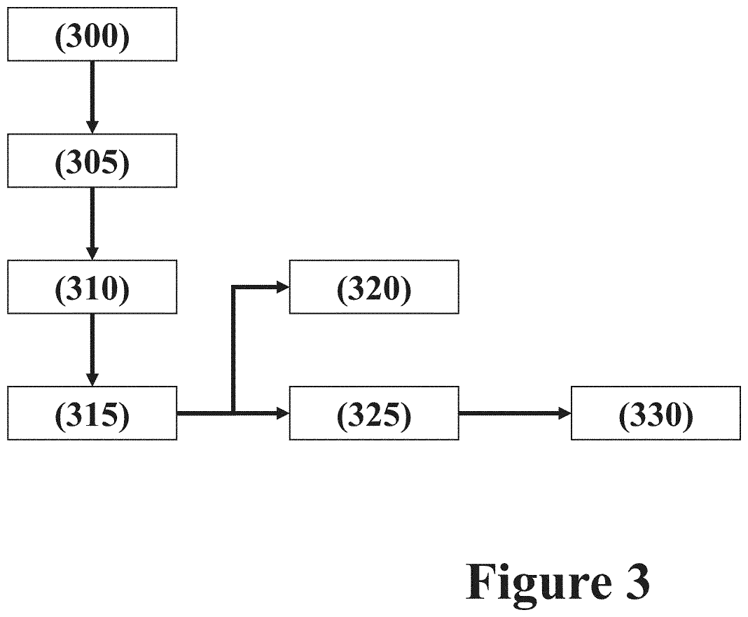

FIG. 4 depicts an embodiment of a system for medical note creation.

FIG. 5 illustrates an embodiment of a system's prompt mapping.

### DETAILED DESCRIPTION OF THE INVENTION

The creation of diagnostic charts and billing sheets of medical patients can be time consuming and conducive to human error. A method or system for creating a medical note which automatically determines the diagnostic and billing codes, correlates the codes, and automatically identifies billing codes may mitigate some of the inefficiencies.

In some embodiments, the system is a dynamic system that builds a medical note in the intuitive order and language of a physician and then inserts the medical note into the EHR. The medical note can be created by the physician during the interview with the patient, the same way that a physician would create a note in an EHR by typing, dictating, or using a template. The system may need no typing, dictating, or template. In some embodiments, the various aspects of the medical note are linked, and the system automatically provides diagnostic and billing codes while the clinician creates the note.

In some embodiments, the system prompts the medical professional to review prompts for selection. In some embodiments, the system receives a selection of a data point. In some embodiments, after the system receives a selection of a data point, the system determines another set of prompts based on the selection of the data point. In some embodiments, the system displays the new set of prompts. In some embodiments, the medical professional is prompted to review the new prompts. In some embodiments, the system receives a selection of another data point. The system may determine a diagnostic code based on the selected data points, wherein the diagnostic code becomes increasingly more specific as more data points are selected. The system may follow the same procedure for further diagnostic points, further refining the diagnostic codes. In some embodiments, the system creates a medical note, with four major parts being: History of Present Illness (HPI), Physical Exam (PE), Diagnosis, and Plan. In some embodiments, the clinician creates the medical note by continuing to make choices from the options the system displays. In some embodiments, during the Plan section, the clinician chooses from among tests and treatment options. In some embodiments, the system automatically determines billing codes matching the selected tests and treatment options. In some embodiments, the four sections of the medical note are linked. The clinician may input data in one section, and the system may auto-populate the other sections automatically where appropriate.

In some embodiments, there are multiple data points in various sections of the medical note that are linked to data points in other sections, in order to arrive at the most accurate diagnoses, and the most accurate diagnostic and billings codes. In some embodiments, the linking ensures that the final note is consistent throughout, avoiding human errors by eliminating touch points. In some embodiments, the diagnosis codes are ICD 10 diagnosis codes.

Past methods of documenting medical data and later translating the data for diagnosis and billing are time consuming and error conducive through the large amount of repetitive documentation and abundance of inapplicable fields. In some embodiments, the system provides a medical physician and other clinicians with a specialized interface for collecting medical data for more efficient data collection, medical diagnosis, and billing. In some embodiments, auto-population methods are utilized to ensure a medical physician or other medical professional does not have to repetitively insert the same information. The auto-population aspect of the system may recommend the previous inserted data for the same prompt or a similar prompt later in the data intake process. Furthermore, in some embodiments, deliberate data intake mapping allows the physician or medical professional to only be given pertinent options related to previously inserted data. Therefore, the medical professional is not barraged with options that do not relate to the professional's specialty or the topic of the concern.

In some embodiments, data intake mapping prompts the medical professional to document general information from the patient. In some embodiments, the system then processes the inserted data to provide further boxes to be filled out to gain more specific information on the relevant data. Therefore, if a patient provides very general symptoms at the initial intake step, the system can prompt the medical professional to intake data more specific to the flagged symptoms and issues to provide a more specific and on-point diagnosis and billing summary. In some embodiments, the system matches the intake data to the correlating diagnosis codes and billing codes. In some embodiments, as the intake data and prompts become more specific, the diagnosis codes and billing codes also become more specific and more precise. The added specificity can lead to more accurate and efficient data documentation for both diagnosis and billing; therefore, aiding in both treatment of patients and billing.

In some embodiments, the initial page for intake and the entire process for creating the medical note is specialized according to the specialty of the physician. In some embodiments, the initial page is specialized depending on the known medical history of the patient. In some embodiments, the process for creating the medical note is specialized depending on the known medical history of the patient. In some embodiments, the initial page is general, and the page specificity accrues through the insertion of data.

In some embodiments, the system's intake of data causes only relevant data points to become prompts for further insertion. For example, in some embodiments, if the physician indicates the patient has a leg ulceration, the system prompts the physician to insert or select data relating to the type of ulceration and the location of the ulceration. For example, in some embodiments, the selection of leg ulceration without the selection of another general issue would bypass any further prompting of selections or insertions of data related to irrelevant data including prompts related to gynecology, urology, or other non-related conditions. The intake mapping can allow for medical professionals to be faced with more detailed prompts on inserted data and spend less time reviewing possible selections related to irrelevant diagnostic conclusions.

In some embodiments, the system intakes data automatically or manually. In some embodiments, data is collected from an intake form. In some embodiments, data is manually inserted by a medical professional. In some embodiments, the collected data includes manual and automatically collected data. In some embodiments, the collected data is matched to correlated diagnosis codes and correlated billing codes. In some embodiments, the collected data, diagnosis codes, and billing codes are processed by the system. In some embodiments, the system creates a final medical note. In some embodiments, the medical note is in paragraph form. In some embodiments, the medical note is in bullet form. In some embodiments, the user of the system is given the option to have a paragraph form medical note or a bullet form medical note. In some embodiments, the system compiles all of the relevant diagnosis codes. In some embodiments, the system compiles all of the relevant billing codes.

In some embodiments, components of the system are linked throughout the process of data intake and medical note creation. In some embodiments, the linked components ensure efficiency through auto-population. In some embodiments, the linked components ensure specificity, depth, and complexity through intake data mapping. In some embodiments, the linked components with embedded diagnostic codes ensure precise automated diagnoses, matching history of present illness, matching physical exam, and matching plan, resulting in a consistent medical note.

In some embodiments, different medical selections cause the system to provide different data prompts for the medical professional. In some embodiments, different medical selections cause the system to remove certain data prompts from the next intake step to add efficiency. In some embodiments, the system requires further data insertion if a certain prompt is selected. In some embodiments, if an injury is selected, the system prompts the medical professional to select the type of injury and the location. In some embodiments, once an injury is selected, the system provides an opportunity to select associated signs and symptoms along with pertinent medical conditions that may affect the injury, or which the injury may affect, such as diabetes, heart disease, or autoimmune disease. If such a condition is selected, the system may add the diagnosis code of that condition or list a new code that specifically describes the combination of the two conditions. (e.g. A leg ulcer may have diagnosis code X, while insulin-dependent diabetes may have a diagnosis code Y, but the two together—diabetes with leg ulceration, may have a totally different diagnosis code Z. The mapping comes from other sections of the note, including Chief Complaint and Physical Exam). In some embodiments, if a certain symptom is selected, the system prompts the medical professional to review further symptom selections to provide a more detailed and complete diagnosis.

In some embodiments, the system tracks the location of an issue on the body for auto-population or suggestion for further data intake. For example, if the medical professional inserts data for an x-ray of the left leg, when the medical professional goes to select treatment through providing a cast, the system will pre-populate left leg.

In some embodiments, the system intakes data for the HPI and PE, then prompts a user to make selections to receive data for the Diagnosis section. In some embodiments, the intake data auto-populates portions of other sections. In some embodiments, a singular data point populates multiple sections or parts.

In some embodiments, the exam information is inputted without prompting. In some embodiments, the exam information is received through prompting. In some embodiments, portions of or all of the exam information can be populated through intake of the diagnosis through backward flow. In some embodiments, the exam information is interlinked with other data related to different sections or parts of the medical note for efficiency and accuracy.

In some embodiments, the interlinking of the data ensures the data matches throughout the medical note.

In some embodiments, the system recognizes the relationship of different treatments and tests for each medical condition. In some embodiments, the system prompts the medical professional to also select the related treatment or test for that condition. In some embodiments, the system automatically inserts the related treatment or test into the data. In some embodiments, the system automatically inserts the related treatment or test with the correct diagnosis code and billing code to provide accurate diagnosis data and precise billing.

In some embodiments, the system continuously updates the diagnosis codes from the Chief Complaint section of the note.

In some embodiments, the system continuously updates the diagnosis codes from the Physical Exam section of the note.

In some embodiments, the system continuously updates diagnosis codes. In some embodiments, diagnosis codes are continuously tabulated. In some embodiments, the diagnosis codes become increasingly more specific throughout the intake process. In some embodiments, diagnosis codes are linked throughout the process.

In some embodiments, the system continuously updates billing codes. In some embodiments, billing codes are continuously tabulated. In some embodiments, the billing codes become increasingly more specific throughout the intake process. In some embodiments, billing codes are linked throughout the process.

In some embodiments, the system can determine codes from the exam. In some embodiments, the system determines the diagnostic codes and billing codes without requiring the user to manually search for the codes. In some embodiments, the system determines the codes without displaying the ICD-10 or CPT code manuals.

In some embodiments, the system determines diagnostic codes throughout the data intake process, becoming more refined and specific as more data is collected. In some embodiments, the system determines the billing code from the Plan section. In some embodiments, upon the compiling of data for the Plan section, a billing code is determined and displayed. In some embodiments, multiple diagnostic codes are displayed before a single billing code is determined. The billing code may be a CPT code. The diagnostic code may be an ICD-10 code. In some embodiments, the system determines these codes without the user manually searching a code manual or directory.

In some embodiments, the system attaches information to the chief complaint. In some embodiments, the system builds on the initial chief complaint. In some embodiments, the system intakes the initial chief complaint, processes the chief complaint, and outputs prompts for the medical provider to review to provide more specific and detailed data.

In some embodiments, the system prompts include but are not limited to the selection of conditions, diseases, symptoms, types of pain, onset, duration, severity, associated signs and symptoms, and other pertinent medical conditions.

In some embodiments, the system provides a tree of intake flow. In some embodiments, the system provides different intake routes or intake flow depending on previously inserted data.

In some embodiments, each condition, injury, or illness has a different prompt or intake flow. The flow may have varying lengths or tangents. In some embodiments, the system allows the user to navigate from one condition flow to another regardless of whether the conditions are related or tangential to one another.

In some embodiments, the system includes pre-designated intake flows and mapping to insure the most efficient, detailed, and specific diagnosis and billing.

In some embodiments, the system is not an EHR. In some embodiments, the system is a dynamic system that builds a medical note in the intuitive order and language of a physician and then inserts the medical note into the EHR. The medical note can be created by the physician during the interview with the patient, the same way that a physician would create a note in an EHR by typing, dictating, or using a template. In the system there may be no typing, dictating, or template. In some embodiments, as the physician interviews the patient, the physician chooses from pre-written clinical options that proceed along an algorithm to diagnose and treat a specific condition in a specific medical specialty. In some embodiments, the options appear as the physician makes choices along the algorithm. In some embodiments, as the physician chooses an option, the diagnosis becomes more refined and the diagnostic ICD 10 codes are automatically listed. In some embodiments, the physician proceeds in the same manner to create the rest of the note, including the Physical Exam and the Plan sections, during which the procedure billing codes (CPT codes) are automatically determined and listed.

In some embodiments, the medical note incorporates data received from the EHR and then sends the incorporated data back to the HER. The system may integrate the incorporated data into specific locations of the EHR. For example, this may occur as a diabetic exam is performed. In some embodiments, the EHR incorporates this linked data into the necessary sites, resulting in the linked data appearing to have been fully created inside the EHR. This linking process may meet “touch points” requirements the physician needs to document in reporting proper use of the EHR to the government.

In some embodiments, the first unique aspect of the system is that the medical note is completed on one webpage. In most EHRs, the physician has to constantly click from one page to another to complete various sections of the medical note. In some embodiments, the system greatly reduces the number of clicks by focusing everything on one page. The one-page aspect can greatly reduce the number of clicks required to create a note and does not exist in typical EHR programs.

Secondly, the system may automatically list the most accurate diagnostic ICD 10 codes and CPT billing codes. In all EHRs, the biller or the physician has to manually choose from among the 68,000 ICD 10 codes and the over 10,000 CPT codes for each patient. In some embodiments, the system saves time and money for the reduced effort, and increases revenue by automatically finding the most accurate codes for a particular condition and/or procedure.

In some embodiments, the linkage of various parts of the medical note is another unique feature of the system, which saves anywhere from 5 to 25 clicks per note. For example, once the physician chooses the location of an injury in one part of the note, the system can auto-populate in the rest of the note wherever a location is needed (e.g. in the physical exam section, in ordering an X-ray, in placing a cast, etc.). In some embodiments, Associated Signs and Symptoms and Pertinent Medical Conditions are also linked with every medical condition and appear as options as the physician creates the note. For instance, if a patient comes in with knee pain, but also has diabetes and arthritis, the system may link these conditions and may automatically refine their associated codes, which change with each added condition. In some embodiments, the system provides deeper context, additional diagnoses and treatment options, along with their automated diagnostic and billing coding.

In some embodiments, other automated linkage occurs throughout the medical content of the system, which greatly reduces the number of clicks and time, and increases physician income. For example, the diagnosis may be linked to the other parts of the note, especially the physical exam. In all other EHR systems, there is only one generic physical exam section that is used for all types of diseases and conditions. In some embodiments of the system, the physical exam is customized for each medical condition, so that an Orthopedic Surgeon examining an elbow will not have the same focus in the physical exam as a Gynecologist or a Dermatologist. The physical exam may be very specific and detailed for that condition. In some embodiments, the physical exam documents specific information, and uses appropriate wording and ICD 10 and CPT codes, that are required by insurance companies to approve a claim at its highest level of reimbursement. In some embodiments, the system allows the physician to change from the condition-specific physical exam to the general “all” exam without leaving the one-page format, allowing more than one condition to be easily documented.

In some embodiments, the medical note includes sections or parts. In some embodiments, the medical notes include a History of Present Illness (HPI) section, a Physical Exam (PE) section, a Diagnosis section, and a Plan section. In some embodiments, the sections are linked, which allows for one data point selection to populate data in multiple sections instead of requiring the user to input the data multiple times. In some embodiments, the linking of the Diagnosis section and the Physical Exam section allows for more efficiency and a more specific diagnosis code. The linking may eliminate conflicting data on the medical note and may provide timelier and more efficient medical note creation. For example, the system may receive input of the left ankle being the injury location. In this embodiment, the input of the left ankle may occur only once, but the system may auto-populate multiple sections of the medical note with the singular data point selection. In some embodiments, the data point selections carry through to any pertinent section or part.

In some embodiments, the system arrives at the diagnosis codes through a totally different manner than an electronic health record. In some embodiments, the system arrives at the most specific diagnosis codes by dynamically linking and mapping data from all parts of the medical note, ensuring the highest level of specificity with less time and energy on the part of the clinician. In some embodiments, the system bypasses the need to search for codes as the system automatically determines the codes.

In some embodiments, the embedding of data and linking of sections allows the system to provide specific diagnostic codes without the need for the user to search the ICD 10 codes manually. In some embodiments, the diagnostic codes and billing codes are automatically determined through the embedding of data related to data point selection. In some embodiments, as more data points are selected, the determination of diagnostic codes becomes more refined and precise. Conditions and medical knowledge may aid in the automated determination of diagnostic codes.

In some embodiments, other linkages include customizing billing and CPT coding to the specific insurance company requirements (e.g., Medicare using supply codes). Another example: Protective CPT & Physical Exam components may be linked to optimize reimbursement and avoid an audit. For example, follow-up visit codes can be linked to follow-up office visits, and NP codes can be linked to New Patient visits, in combination with certain sections of the note i.e., New Patient exam includes Past Surgical History (PSH), Past Medical History (PMH), Review of Systems (ROS), and Social History (SH) while follow-up exam only uses PMH and ROS.

In some embodiments, the data including linkages make up the “medical content.” In some embodiments, the medical content is not extracted from anywhere, the content exists in the system. In some embodiments, the medical note is created by the physician or other clinician as the clinician interviews the patient, the same way medical notes are normally done.

In some embodiments, the system contains hundreds of thousands of lines of medical content in all specialties.

In some embodiments, the system creates the medical note outside of the architecture of the EHR. In effect, the system can bypass that section of the EHR. In some embodiments, after the medical note is completed, the system inserts the medical note, along with the diagnostic and billing codes, back into the appropriate places in the EHR. In some embodiments, the insertion is what makes the system universally applicable to all EHRs. Because the architecture of most EHRs can be inadequate for creating the quality of medical note that physicians require, the system may create its own architecture, with its vast library of medical content, to create the medical note outside of the EHR. In some embodiments, the system integrates with the EHR at certain points to insert the medical note, codes, and other information back into the EHR.

In some embodiments, the system integrates with medical content and billing system data from an electronic health records (EHR) system. In some embodiments, the system is able to integrate with the data from any and all EHR systems. In some embodiments, the specific and condition-specific content is isolated for a specific purpose.

In some embodiments, the system reduces the number of clicks and interactions necessary for a medical worker to create a medical note or medical report.

In some embodiments, diagnosis codes and billing codes are embedded with the medical content. In some embodiments, the embedded codes allow for the listing of the codes when creating the medical note. The embedding of codes may lead to less human error when compiling billing data as the billing codes are embedded with the medical content to ensure the correct billing code is attached to the correct medical content.

In some embodiments, the relevant diagnostic and billing codes are automatically linked to the medical note. This may allow the doctor to continue viewing a single page. In some embodiments, containing the information to a single page saves time for the clinician by eliminating numerous points of contact with the medical note. In some embodiments, the diagnostic and billing codes auto-populate to the appropriate locations in the EHR.

In some embodiments, the physician's note consists of several parts, which include the Chief Complaint, the History of Present Illness (HPI), the Past Medical History (PMH), the Past Surgical History (PSH), the Review of Systems (ROS), the Physical Exam (PE), the Diagnosis, and the Plan sections. In some embodiments, the sections are linked in order to create a fully inclusive medical note that encompasses all needed diagnosis codes and billing codes.

In some embodiments, the Chief Complaint, History of Present Illness, Review of Systems, the Family and Social History and the Physical Exam are linked to contribute to the final Current Procedural Terminology (CPT) coding. In some embodiments, the medical-decision making is also included in the medical note.

In some embodiments, the system enables the clinician to create a medical note by clicking on choices that branch forward as each choice is made. In some embodiment, with each choice, the diagnosis is refined and made more specific. In some embodiments, as the clinician makes the choices, ICD10 diagnosis codes are automatically gathered by the system to refine and make them more specific, which increases the specificity of the medical data as well as the reimbursement. In some embodiments, the system further intelligently links sections of the note to build upon the clinical note and automatically populate information already inserted in previous sections, thus saving the physician clicks, time, and errors.

In some embodiments, the system takes information from the HPI and pulls the information into the Diagnosis and Plan sections, further refining CPT coding and the Plan for testing and treatment. In some embodiments, information in the Location section automatically populates in the HPI, the Physical Exam, and the Plan sections, saving clicks and further automatically refining the CPT and ICD10 coding. In some embodiments, information and context provided by the Associated Signs and Symptoms, Additional Diagnoses, and Pertinent Medical Conditions from the HPI section links to the rest of the note to provide context, which affects not only clinical judgment but the level of difficulty of the clinical case and the specificity of the ICD10 and CPT coding, all of which increase reimbursement. In some embodiments, specific linking that does not exist in any other EHR includes Context, Associated Signs and Symptoms, and Past Medical History. In some embodiments, context further refines the ICD10 codes and/or gives additional ICD10 codes. In some embodiments, Associated Signs and Symptoms gives additional ICD10 codes and shows the increasing complexity of the patient. Past Medical History can link the ICD10 codes and can provide information showing the increased complexity of the patient thus increasing the reimbursement to the physician.

In some embodiments, the CPT codes and ICD-10 codes are automatically calculated from what the clinician chooses while creating the note. In some embodiments, the clinician or biller does not have to search a list to manually pick any codes.

In some embodiments, a clinician can choose to have defaults in the Plan section that save those particular choices for a specific condition for each clinician. With one click a clinician can choose the default, and the system may generate the tests and treatment for that condition and all the CPT billing codes for that patient. In some embodiments, the refined type of defaults have not yet been seen in any other EHR.

In some embodiments, the system automatically generates all ICD10 diagnosis and CPT billing codes directly from the clinical note, which eliminates discrepancy between the clinical note and the coding, a major issue in Medicare and insurance auditing. All the codes can automatically match the clinical note to their highest level of specificity, increasing physician revenue in multiple ways. Increased specificity of the codes leads to increased revenue; decreasing costs due to decreased biller and/or clinician time; decreasing billing errors, which avoids resubmissions; and the ability to increase patient load per clinician per day, which increases revenue, can all be accomplished through the system.

In some embodiments, the system utilizes an automated, complex linking process, and the detailed documentation creates a comprehensive medical note, increasing the value and detail of the clinical note, avoiding typing or dictating, decreasing time and clicking, avoiding clinical errors and patient morbidity, avoiding billing errors, and increasing revenue.

In some embodiments, the medical note automatically creates a special code (e.g., a “G-code”) linked to treatments of particular diseases. The data points to the special codes may populate the EHR. This may satisfy the “minimal use” requirements of government regulations to count towards certification of the EHR.

In some embodiments, the system includes a database of prompts related to medical subject matter pertinent for diagnostics and billing in the medical field. In some embodiments, the system further includes a database of diagnosis codes. In some embodiments, the system further includes a database of billing codes. In some embodiments, the system displays prompts for a medical professional to review and possibly select data points. In some embodiments, the selection of data points initiates the searching, linking, or embedding of a diagnosis code and a billing code. In some embodiments, as more selections are made, the more refined and specific the diagnosis codes and billing codes become. In some embodiments, the system processes the selection of data points and determines what prompts to display next to the medical professional for review. In some embodiments, the system automatically selects certain data points based on previous selections. In some embodiments, the system interlinks the prompt database, the diagnosis code database, and the billing database. In some embodiments, the data points, diagnosis codes, and billing codes are compiled, and a medical note is created with the data being displayed to the medical professional in either paragraph form or bullet form. In some embodiments, the system inputs the medical note into an electronic health records system.

In some embodiments, the system is fully automated. In some embodiments, the system automatically determines the next prompts to display by the use of a processor. In some embodiments, the system automatically determines the diagnosis codes and the billing codes by the use of a processor. In some embodiments, the system automatically populates certain fields by the use of a processor. In some embodiments, the system automatically selects data points by the use of a processor.

In some embodiments, the system displays the data selection and prompt options on a singular page, window or screen which is an improvement from existing EHR systems that requires navigation through many screens, pages, or windows, often with irrelevant information displayed or requiring to be input. In some embodiments, the user is able to navigate from specific conditions of one injury/illness to the all choices feature of the system. For example, the user may originally select one injury and select specific information for that particular injury, disclosing location and other pertinent information, then the user can navigate to the “all” section in order to input information related to a different injury. In some embodiments, the system affords the user the ability to navigate from one section to the next multiple times while staying on a singular page. In some embodiments, the intake of a large amount of data occurs on a singular page.

In some embodiments, the system intakes the chief complaint (100), history of present illness (105), review of systems (110), and the past family and social history (115). In some embodiments, the data is compiled into a medical note (120). In some embodiments, the medical note is embedded with diagnosis codes (125) and billing codes (130). In some embodiments, the chief complaint (100), history of present illness (105), review of systems (110), and the past family and social history (115) are manually inserted by a medical professional. In some embodiments, the insertion is aided by the system's auto-population features. In some embodiments, the insertion is aided by the system's designated intake mapping. The system can flow in the opposite direction. In some embodiments, the user inputs the diagnosis and the system auto-populates the fields to aid in the creation of a medical note.

In some embodiments, the system intakes the chief complaint (200), history of present illness (205), review of systems (210), and the past family and social history (215). In some embodiments, the data is found in an EHR with diagnosis codes (220) (225) (230) and billing codes (235) (240) (245). In this embodiment, the information in the bisectional area is analyzed and determines which diagnosis codes and billing codes are put into the medical note.

In some embodiments, the system intakes physician-entered medical information into an EHR system (300). In this embodiment, the system intakes a request from physician or a billing associate for a medical note (305). In this embodiment, the system intakes relevant data including diagnosis, diagnosis codes, and billing codes (310). In this embodiment, the system compiles the data into a medical note (315). In this embodiment, the system compiles diagnosis codes for medical diagnosis (320). In this embodiment, the system compiles the billing codes for a billing summary (325). In this embodiment, the billing summary is utilized to bill the client (330).

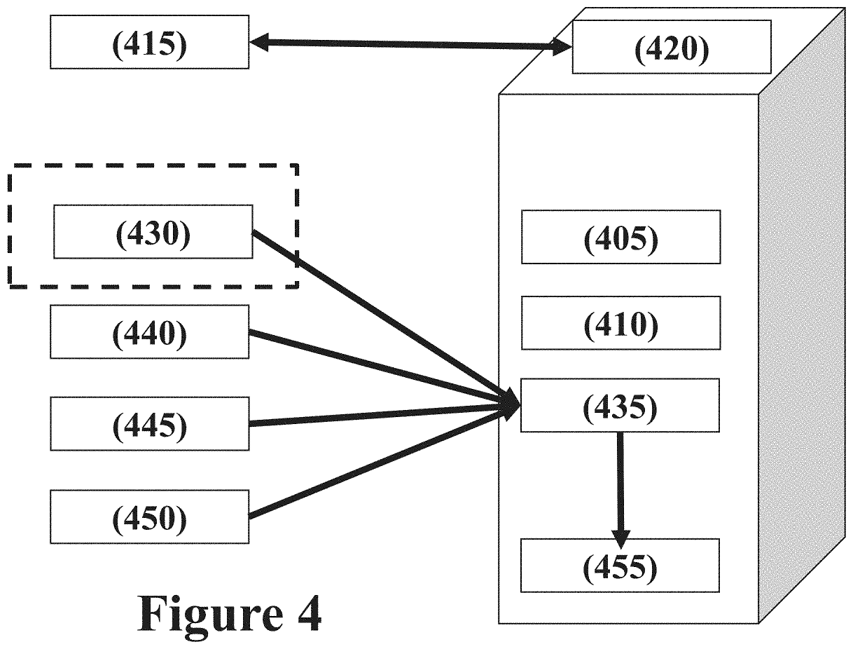

FIG. 4 depicts the system environment of one embodiment of the processing system. In some embodiments, the module (420) includes stored codes, system memory, stored diagnosis (405), character recognition application (410), a processor (435), and a database of algorithms. In some embodiments, the application intakes data (430), then relays the data to the processor (435). In some embodiments, the processor receives data from other sources (440) (445) (450) to interpret the data. The other sources may be manual input or a variety of other sources. In some embodiments, the processor intakes the data, recognizes relevant fields, finds relevant codes, and embeds the relevant codes with the correlating field data. In some embodiments, the data and embedded codes are used to create a medical note (455). The module may also send the billing information for efficient billing.

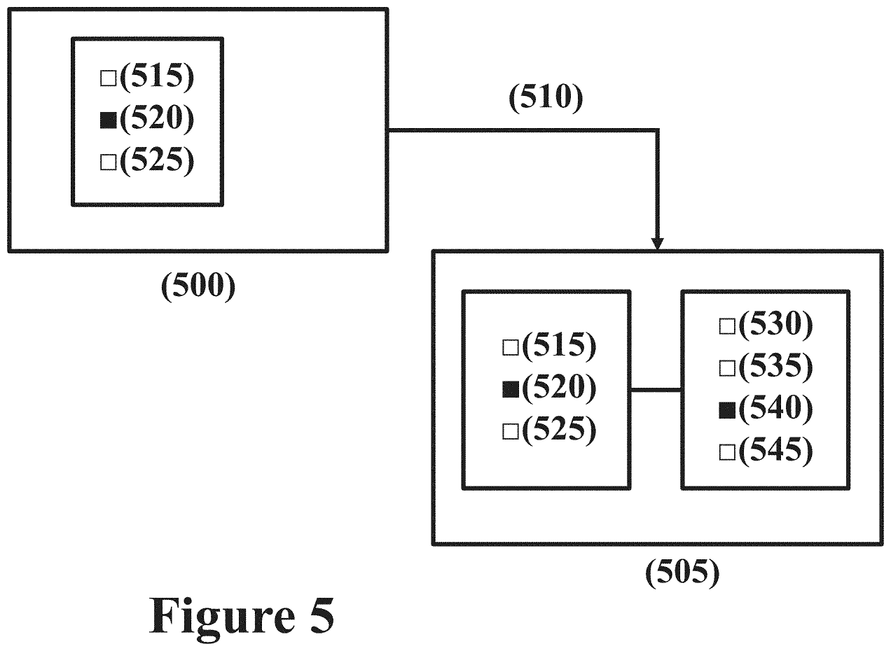

FIG. 5 depicts one embodiment of the system's flow. In some embodiments, the system displays a first set (500) of various prompts (515) (520) (525) for a medical professional to review. In some embodiments, the system intakes data point selections (520), when the prompt relates to the patient's illness. In some embodiments, once the first set (500) of prompts is reviewed, the system transitions (510) to a second set (505) of various prompts (530) (535) (540) (545) related to more specific matters pertaining to the initial data point selection (520). In some embodiments, the system repeats the steps until a medical note is completed. In some embodiments, the second set (505) of various prompts include only relevant prompts to the initial selection (520). In some embodiments, a billing code is determined based on the selection in the first set (500) and a more refined billing code is determined with the selection in the second set (505).

In one example of an embodiment the present system, a physician's note may include 1) Chief Complaint, 2) Physical Exam, 3) Diagnosis and 4) Plan, and the physician may be presented with the option to start at one of these for parts of the physician's note. If, for example, the physician is presented the option to start at parts 1-3, the information entered, such as location of the chief complaint, or other conditions/context entered into data prompts, is transferred and used in all parts of the note. For example, if in Chief Complaint, the physician chooses the location of the chief complaint, that location will be transferred to all other parts of the note, and the physician will only be presented with options relevant to the particular location chosen to all parts of the note, limiting the number of clicks necessary for the physician to make to complete the note. For example, in the Plan portion of note, large groups of CPT codes exist for various procedures. There may be an X-ray “all” box that has every CPT code for the (X-ray) entire body listed out with every “view.” There may be another box with every “I&D” for the entire body with attached code and specific wording needed by insurance companies. If the physician lists the location in 1, Chief Complaint as “knee” the system will shorten the choices in the X-ray and I&D boxes for only those pertaining to the knee. The physician may still have the option to get back to all options to check, for example, chest X-ray, if so desired. However, because of the dynamic linking of content between the “Chief Complaint” and the other portions of the note, the system presents or predicts only the relevant codes to “location” chosen in Chief complaint. The physician, thus, does not have to scroll through hundreds of choices.

As a further example, if the physician chooses the option for edema present in the Physical Exam-related prompts, this option is transfer to the Diagnosis and Plan parts of the note, as well as backward to Chief Complaint. If, for example, family history of a congenital deformity is an option that is selected during any part of the note, a separate diagnosis will be added to the Plan or the Diagnosis part of the note, and it may also further define the ICD-10 coding. For example, the ICD-10 code for a gout diagnosis may be linked with multiple ICD-10. An ICD-10 code may exist for gout without other medical conditions, but a different ICD-10 may be defined for gout with other medical conditions, such as end stage renal disease.

The system, may, thus present the option of beginning the note at any portion of the note, such as Physical Exam, and will automatically make changes and/or present choices in the other portions of the note, such as Chief Complaint, Diagnosis, and Plan, that relate only to the newly selected condition chosen in Physical Exam, for example. The system will also auto-populate all portions on the note for what needs to or should be said to the insurance company, making the note more consistent.

In some embodiments, the choices made in the earlier parts of the note, such as Physical Exam will pull patent education or anticipatory guidance based on the conditions selected, as well as the other Plan section choices related to the choices made in Physical Exam, or other sections. For example, if the Physician included “cellulitis” in the Physical Exam section of the note, the system may pull patient education data written about cellulitis into the plan section.

In yet other embodiments, a physician may be presented with a “location” graphic, for example, a graphic of a body, wherein the physician is able to click a portion of the body, which, is linked to and, in turn, presents the physician with all codes related to that location on the body.

In some embodiments, the embodiment further includes sending, receiving, or storing data, instructions, or both upon a computer-readable medium. Methods disclosed above may be accomplished by one computer or may be accomplished through a plurality of computers, and the method should not be construed as one or the other. The methods may be implemented in hardware, software, or an amalgamation of both. The systems, methods, and procedures disclosed herein can be embodied in a programmable computer, computer-executable software, or digital circuitry. The software can be stored on computer-readable media. Some examples of computer-readable media can include a RAM, ROM, floppy disk, hard disk, flash memory, memory stick, removable media, optical media, magneto-optical media, CD-ROM, or any other viable form. Digital circuitry can include, but not limited to, integrated circuits, building block logic, gate arrays, field programmable gate arrays, or any other viable form. In some embodiments, the method may be reordered, changed, additional steps added, steps removed, steps combined, and otherwise modified. In some embodiments, the steps are automated. Chronological wording such as first, second, third, and so forth should not be viewed as limiting, but instead as one possible embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A medical note system comprising:
one or more processors; and
one or more memories operatively coupled to at least one of the one or more processors and having instructions stored thereon that, when executed by at least one of the one or more processors, cause at least one of the one or more processors to:
receive instructions for an initial prompt;
display the initial prompt for a user to interact with the initial prompt;
recognize selection of an initial data point, wherein the initial data point includes information relevant for determining diagnosis and billing;
determine a diagnosis code matching the recognized initial data point;
determine a second prompt based on selection of initial data point, wherein the second prompt relates to more specific information related to the initial data point coming from the initial prompt;
display the second prompt;
recognize selection of a second data point, wherein the second data point includes information relevant for determining diagnosis and billing;
determine a second diagnosis code matching the recognized second data point, wherein the second diagnosis code relates to the more specific second data point; and
create a medical note, wherein the medical note includes the second diagnosis code and sections, wherein the sections are structured data sets from the selection of data points;
wherein the sections are interlinked to allow for a single selection to populate the structured data for multiple sections, wherein the interlinking is configured to provide specific diagnostic codes and specific billing codes.

2. A medical note system as in claim 1, wherein the medical note further comprises the initial data point and the second data point.

3. A medical note system as in claim 1, further comprising auto-populate a third prompt based on the selection of a previous data point.

4. A medical note system as in claim 1, wherein a determination of the second prompt is determined based on a designated intake mapping structure.

5. A medical note system as in claim 1, further comprising input the medical note into an electronic health records database.

6. A medical note system as in claim 1, wherein the diagnosis codes are ICD 10 diagnostic codes.

7. A medical note system as in claim 1, wherein the medical note is condition-specific.

8. A non-transitory computer-readable storage medium storing program instructions computer-executable to perform, comprising:
receiving instructions for an initial prompt;
displaying the initial prompt for a user to interact with the initial prompt;
recognizing selection of an initial data point, wherein the initial data point includes information relevant for determining diagnosis and billing;
determining a diagnosis code matching the recognized initial data point;
determining a second prompt based on selection of initial data point, wherein the second prompt relates to more specific information related to the initial data point coming from the initial prompt;
displaying the second prompt;
recognizing selection of a second data point, wherein the second data point includes information relevant for determining diagnosis and billing;
determining a second diagnosis code matching the recognized second data point, wherein the second diagnosis code relates to the more specific second data point; and
creating a medical note, wherein the medical note includes the second diagnosis code and sections, wherein the sections are structured data sets from the selection of data points;
wherein the sections are interlinked to allow for a single selection to populate the structured data for multiple sections, wherein the interlinking is configured to provide specific diagnostic codes and specific billing codes.

9. A non-transitory computer-readable storage medium storing program instructions computer-executable to perform as in claim 15, wherein the medical note further comprises the initial data point and the second data point.

10. A non-transitory computer-readable storage medium storing program instructions computer-executable to perform as in claim 15, further comprising auto-populating a third prompt based on the selection of a previous data point.

11. A non-transitory computer-readable storage medium storing program instructions computer-executable to perform as in claim 15, wherein a determination of the second prompt is determined based on a designated intake mapping structure.

12. A non-transitory computer-readable storage medium storing program instructions computer-executable to perform as in claim 15, further comprising inputting the medical note into an electronic health records database.

13. A non-transitory computer-readable storage medium storing program instructions computer-executable to perform as in claim 15, wherein the diagnosis codes are ICD 10 diagnostic codes.

14. A medical note method comprising:
receiving instructions for an initial prompt;
displaying the initial prompt for a user to interact with the initial prompt;
recognizing selection of an initial data point, wherein the initial data point includes information relevant for determining diagnosis and billing;
determining a diagnosis code matching the recognized initial data point;
determining a second prompt based on selection of initial data point, wherein the second prompt relates to more specific information related to the initial data point coming from the initial prompt;
displaying the second prompt;
recognizing selection of a second data point, wherein the second data point includes information relevant for determining diagnosis and billing;
determining a second diagnosis code matching the recognized second data point, wherein the second diagnosis code relates to the more specific second data point;
following a same procedure for further diagnosis points, further refining the diagnostic codes;
creating a medical note, comprising: a History of Present Illness section, a Physical Exam section, a Diagnosis section, and a Plan section, wherein a clinician creates the medical note by continuing to make choices from the options the system displays;
wherein the medical note builds upon a set of medical note data by linking data obtained as a user inputs more data
wherein when receiving input for the Plan section, the data received by the user is chosen from among the tests and treatment options presented, which are linked to other sections of the medical note so that only appropriate options are presented that are relevant to the diagnoses;
automatically determining a billing code matching selected tests and treatment options;
wherein the sections of the medical note are linked, so that if the user inputs data in one section the data is auto-populated in the other sections where appropriate;
wherein the sections are also linked to combine diagnostic and billing data for the medical note to include the most specific diagnostic codes and the most specific billing codes; and
wherein the medical note includes the initial data point, the second data point, and multiple other data points.

15. The medical note system of claim 1, wherein the first and second prompts are on a single page.
